(19) United States
(12) Patent Application Publication
(10) Pub. No.: US 2006/0013410 A1
(43) Pub. Date: Jan. 19, 2006

(54) Mobile-telephone adapters for automatic-noise-reduction headphones

(76) Inventor: Michael J. Wurtz, St. Paul, MN (US)

Correspondence Address: Schwegman, Lundberg, Woessner & Kluth, P.A., P.O. Box 2938, Minneapolis, MN, 55402, US

(21) Appl. No.: 11/111,482

(22) Filed: Apr. 20, 2005

Related U.S. Application Data
(60) Provisional application No. 60/563,716, filed on Apr. 20, 2004.

(51) Int. Cl. H04R 1/10; A61F 11/06

(52) U.S. Cl. 381/74; 381/72

(57) ABSTRACT

One exemplary device, a hands-free adapter for use with ANR headphones, includes a microphone and two stereo-type plugs electrically coupled via a cable. The first stereo-type plug connects to a hands-free jack of a mobile telephone, and the second plugs connects to the audio-input jack of the headphones. The microphone is mounted on the second plug and electrically coupled through the cable and the first plug to a microphone input portion of the hands-free jack. This arrangement allows convenient and unprecedented use of the ANR headphones with the mobile telephone or other suitably equipped communication devices.

### CROSS-REFERENCE TO RELATED APPLICATIONS

This application claims priority to co-pending U.S. provisional application 60/563,716, filed Apr. 20, 2004. The provisional application is incorporated herein by reference.

### TECHNICAL FIELD

The present invention concerns headphones and related circuits, accessories, and methods.

### BACKGROUND

Headphones are used in variety of applications to facilitate private listening of devices, such as stationary and portable stereos, digital video players, digital music players, computers, and so forth. Some of these headphones are equipped with automatic noise reduction (ANR) circuitry. This circuitry automatically cancels or suppresses loud persistent ambient noise within the headphones, allowing users to enjoy an electronically controlled silence or an improved listening experience.

A seminal example of ANR headphones is the Quiet Comfort™ line of headphones from Bose Corporation of Framingham, Mass. Bose recently released a new version of these headphones, Quiet Comfort 2™, which incorporates improvements, such as a fold-flat design for more space-efficient storage and integration of its ANR electronics and battery box into its earcups. (Quiet Comfort and Quiet Comfort 2 are presently believed to be trademarks of the Bose Corporation.) The new version also includes an audio input plug with a built-in audio attenuator. The audio attenuator has a high-low switch to reduce or attenuate the volume of audio signals input to the headphones. The audio input plug is coupled via an insulated multi-wire electrical cable to a standard ⅛-inch male headphone plug, which is compatible with the female audio output of most laptops, and portable video and music players.

Despite these improvements, the present inventor has recognized that the Bose Quite Comfort headphones, as well as other competing ANR headphones, are not readily adaptable for use with two-way communications devices, such as cordless or mobile telephones, or for simultaneous connection to more than one audio source. For example, the Quiet Comfort and other headphones are typically provided with a two-prong-plug adaptor for coupling the headphone plug to the audio output port typically found in commercial airliners. It is also typical to provide an adapter for coupling the ⅛-inch headphone plug to a ¼-inch stereo phone port commonly found on stationary home stereo and entertainment systems. However, none of these adapters allow use of the ANR headphones with two-way communications devices or multiple audio sources.

Accordingly, the present inventor has identified unmet needs to expand the utility and ultimately the value of ANR headphones.

### SUMMARY

To address this and/or other needs, the present inventors devised one or more systems, devices, circuits, and methods for expanding the utility and value of ANR and non-ANR headphones. One exemplary device, a hands-free adapter for use with ANR headphones, includes a microphone and two stereo-type plugs electrically coupled via a cable. The first stereo-type plug connects to a hands-free jack of a mobile telephone, and the second plugs connects to the audio-input jack of the headphones. The microphone is mounted on the second plug and electrically coupled through the cable and the first plug to a microphone input portion of the hands-free jack. This arrangement allows convenient and unprecedented use of the ANR headphones with the mobile telephone.

### BRIEF DESCRIPTION OF DRAWINGS

FIG. 1 is a block diagram of an exemplary system 100 corresponding to one or more embodiments of the present invention.

FIG. 2 is a partial electrical schematic of system 100, corresponding to one or more embodiments of the present invention.

FIG. 3 is a block diagram of an exemplary system 300 corresponding to one or more embodiments of the present invention.

FIG. 4 is an electrical schematic of system 300, corresponding to one or more embodiments of the present invention.

FIG. 5 is an exemplary electrical schematic of a source selector component of system 300, corresponding to one or more embodiments of the present invention.

FIG. 6 is a partial perspective view of an exemplary earpiece-and-connector subassembly 600 for systems 100 and 400, which corresponds to one or more embodiments of the present invention.

### DETAILED DESCRIPTION OF EXEMPLARY EMBODIMENTS

The following detailed description, which references and incorporates the attached Figures, describes and illustrates one or more specific embodiments of the invention. These embodiments, offered not to limit but only to exemplify and teach, are shown and described in sufficient detail to enable those skilled in the art to implement or practice the invention. Thus, where appropriate to avoid obscuring the invention, the description may omit certain information known to those of skill in the art.

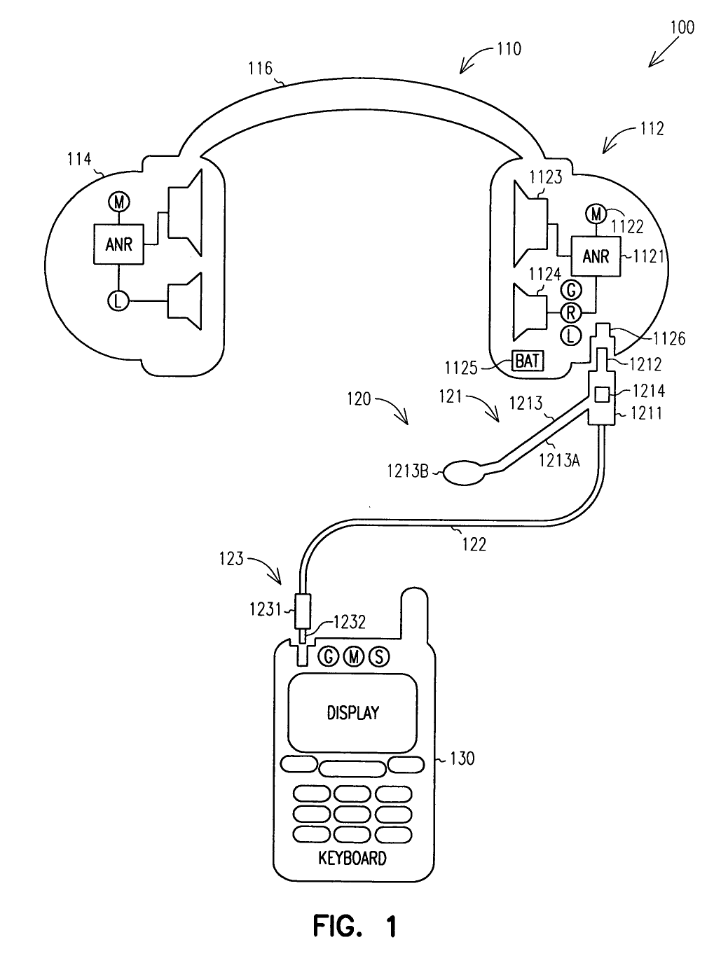

FIG. 1 show an exemplary system 100 corresponding to one or more embodiments of the present invention. System 100 includes ANR headphones 110, an adapter 120, and a communications device 130. System 100 can be sold or offered as a complete system, as separate components, or as subcombination kits. For example, one exemplary kit includes headphones 110 and adapter 120, and another exemplary kit includes communications device 130 and adapter 120.

ANR headphones 110 includes earpieces 112 and 114 and a bridge member 116. Earpieces 112 and 114, each of which take an over-the-ear (circumaural) form in the exemplary0 embodiment, fit over or engage respective ears of a user (not shown). In some embodiments, the earpiece take on-the-ear, in-the-ear, or behind-the-ear form.

Specifically, earpiece 112, which is mechanically connected to earpiece 114 via bridge member 116, includes ANR circuitry 1121, an ANR microphone 1122, an ANR speaker 1123, a non-ANR speaker 1124, a battery compartment 1125, and an audio input jack 1126. In the exemplary embodiment, earpiece 114 is substantially identical to earpiece 112 with the exception of battery compartment 1125 and audio input jack 1126; thus, for sake of brevity, no further description of earpiece 114 is given. Bridge member 116, in some embodiments, folds in half. Also in some embodiments, earpieces 112 and 114 each rotate inwardly (toward the region between the earpieces.)

ANR circuitry 1121, which is powered for example by one or more AA or AAA batteries in battery compartment 1125, responds to a range of low-frequency acoustic energy sensed via ANR microphone 1122 by driving ANR speaker 1123 to produce an opposing acoustic signal. The opposing acoustic signal destructively interferes with the low-frequency acoustic energy, thereby reducing its magnitude and improving the clarity of acoustic signals from non-ANR speaker 1124. Speaker 1124 is driven via electrical signals from a right-channel contact 1126R of audio input jack 1126—which includes a ground contact 1126G, a right-channel contact 1126R, and a left-channel contact 1126L—is coupled or couplable to adapter 120. (In the figures, the numerical prefixes for the contacts are omitted.)

Adapter 120 includes a headphone-microphone connector 121, a cable 122 and a device connector 123.

Headphone-microphone connector 121, which in the exemplary embodiment takes the form of three or four-contact male or female stereo plug connector, includes a connector housing 1211; a multi-contact stem or socket 1212; controls 1213; and a boom microphone assembly 1214. Connector housing 1211, formed by molding a durable insulative material, such as plastic, holds multi-contact stem 1212 and supports boom microphone assembly 1213. Multi-contact stem 1212 includes a ground contact region 1212G, a right-channel contact region 1212R, and a left-channel contact region 1212L, which make electrical contact with respective contacts of 1126G, 1126R, and 1126L when connector 121 is properly engaged with audio input jack 126. (The figure omits the 1212 prefix from the contact reference labels.) Controls 1214 include one or more switches, potentiometers, or other devices for muting or adjusting the volume of signals output from connector 121 to headphone 110.

Boom microphone assembly 1213 includes an boom 1213A and a microphone 1213B.

Boom 1213, which can be formed of flexible conduit, extends laterally from connector housing 1211 to support and position (or allow positioning of) microphone 1213 in a region suitable to promote sensing of audible signals from a user wearing headphones 110 equipped with adapter 120. In some embodiments, microphone 1213B is positioned in and/or on connector housing 1211 and boom 1213A is a tube (or wave guide) acoustically bridging all or part of distance between microphone 1213B and a user's mouth. (See, for example, U.S. application 20040062413, published Apr. 1, 2004, which is incorporated herein by reference.)

Microphone 1213B, in the exemplary embodiment, includes an unbuffered electret microphone. This type of microphone is generally suitable for use with microphone preamplifiers typically found in mobile and cordless telephone phones or other devices compatible with hands-free accessories. (Some embodiments also include, within the connector housing, a battery and a high-performance preamplifier for the microphone, thereby improving audio performance over the level provided by the preamp in the communications device.) Microphone 1213B includes positive and negative contacts (not visible in the figure), which are coupled along with the contact regions of multi-stem 1212 to one or more insulated conductors in cable 122. (Some embodiments incorporate a Bluetooth-compatible wireless transceiver and battery within connector housing 1211 to replace cable 1222.)

Coupled electrically to cable 1222 is device connector 122. Device connector 122, which in the exemplary embodiment takes the form of an ⅛-inch (2.5-millimeter) three-conductor stereo plug or socket, includes a connector housing 1221 and a multi-contact stem or socket 1222. Connector housing 1221, formed by molding a durable insulative material, such as plastic, holds multi-contact stem 1222. Multi-contact stem 1222 includes a ground contact region 1222G, a microphone contact region 1222M, and a speaker contact region 1222S., may use a, which make electrical contact with respective contacts of 1326G, 1326M, and 1326S when connector 122 is properly engaged with hands-free jack (or connector) 132 on communications device 130. (The figure omits the 1222 prefix from the contact reference labels.)

Communications device (or system) 130 includes, among other items, an audio output jack 122 and an external microphone jack 124. In the exemplary embodiment, communications device 120 takes the form of a cellular or cordless telephone, with output jack 122 and microphone jack 124 coupled to interface circuitry (not shown) which supports use of a conventional hands-free mobile-phone headset, which includes a microphone and an ear-piece (or headphones). (Hands-free headsets typically include an unbuffered electret microphone that is powered by interface circuitry (not shown) in the cell phone or other type secondary device. In the exemplary embodiment, this interface circuitry is not suitable for boom microphones in aviation headsets.) In some other embodiments, device 130 takes the form of a two-way radio, laptop computer, or other audio source or audio output device, such as a music or video player or other personal listening device. In some of these embodiments, connector 122 is implemented as two separate stereo plugs or connectors for use with communications devices having separate microphone and headphone jacks.

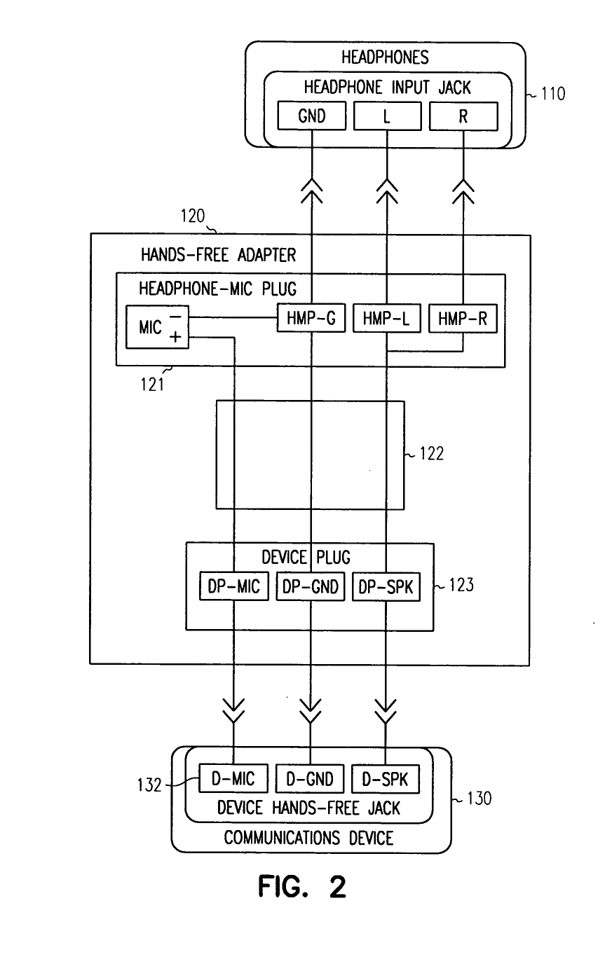

FIG. 2 shows a partial electrical schematic of system 100. The schematic highlights the electrical connections between headphone input jack 1126 and headphone-microphone connector (HMP) 123 and between device connector 123 and device hands-free jack 132.

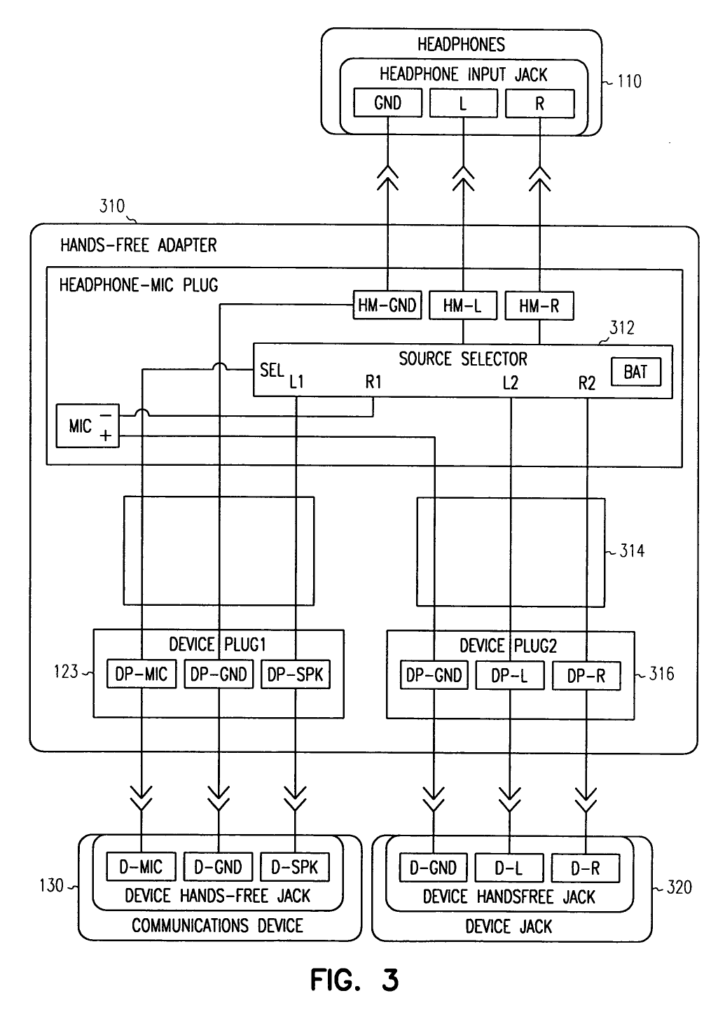

FIG. 3 shows an exemplary headphone system 400 which in addition to previously described headphones 110 and two-way communications device 130, includes a multi-source (or device) adapter 410 and an additional communications device 420. In addition those components already described for adapter 120 in FIG. 1, multi-source adapter 410 includes a source selector 412, a multi-conductor cable 414, and a device plug 416. Source selector 412, which is positioned within the housing of headphone-microphone connector 1211, is coupled via cable 122 to device connector 123 and via cable 412 to device connector 416. Device connector 416, which for example take the form of a 2.5 or 3.5 millimeter stereo plug connector, couples to the audio output jack of communications device 420. Device 420 in some embodiment takes the form of a digital music or video player, such as an iPod music player or other MP3 player.

In operation, source selector 412 normally couples device connector 416 (and thus communications device 420) to the stem of connector 1211 and thus to the audio input jack of headphones 110. However, when device 130 generates a microphone bias signal, for example in response to receiving a phone call and ringing, a microphone bias signal is communicated through cable 122 to selector 412. In response, selector 412, which functions as a break-before-make multiplexer, decouples communications device 420 from the stem of connector 1211 and couples device connector 123 and thus device 130 to stem of connector 1211 and to the audio input jack of headphones 110.

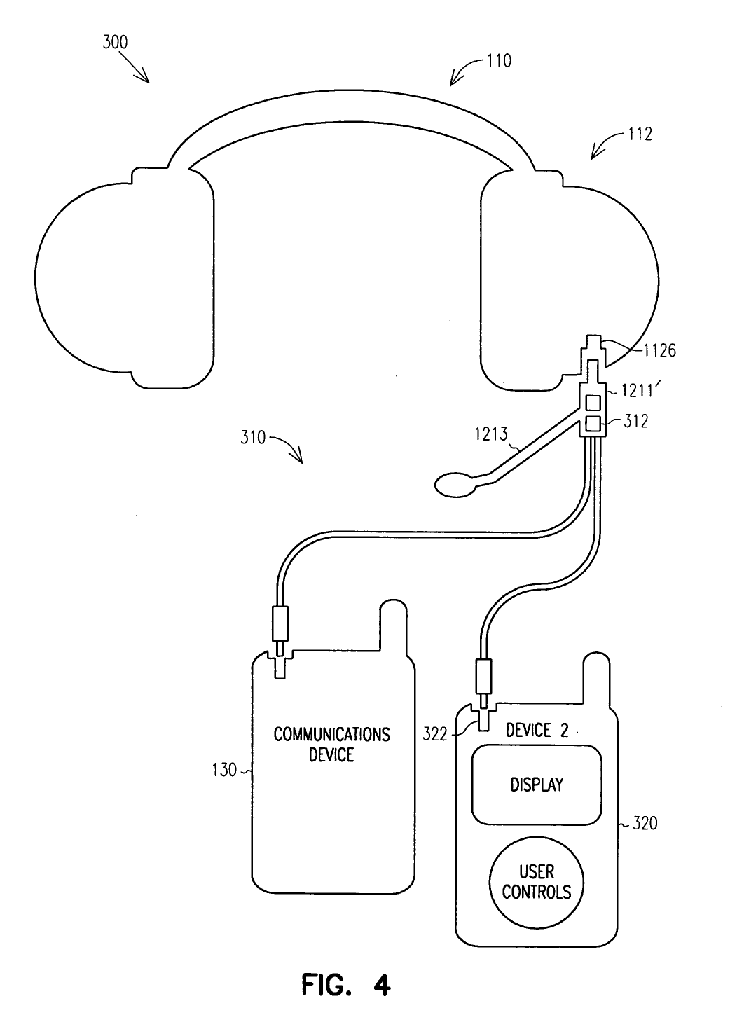

FIG. 4 shows an exemplary electrical schematic for system 400, which like FIG. 2 highlights connections within adapter 410 and connection of adapter 410 to headphones 110 and devices 130 and 420.

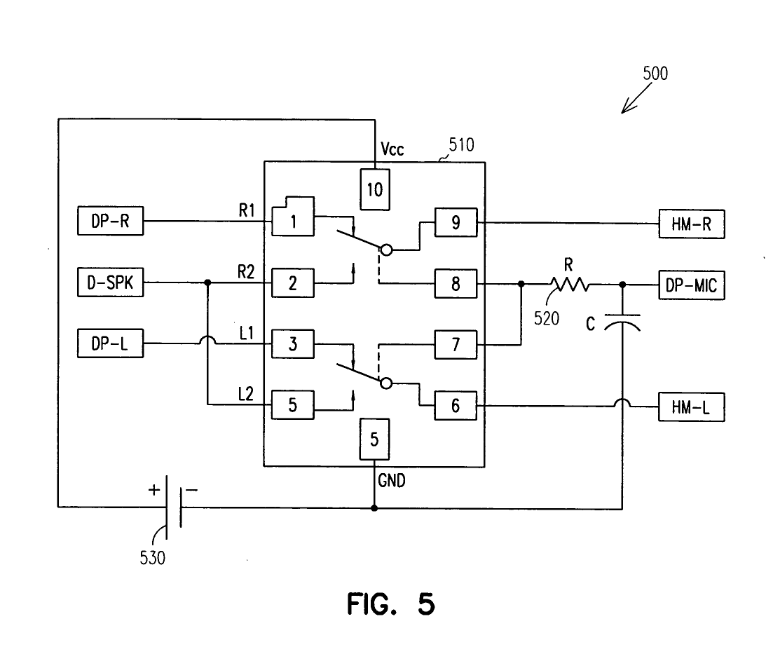

FIG. 5 shows an exemplary implementation of source selector 412 in the form of circuit 500. Circuit 500 includes dual SPDT (single-pole-double-throw) analog switch 510, a low-pass RC filter 520, and a battery 530. One example of a suitable analog switch is part number FSA2267 from Fairchild Semiconductor of South Portland, Me. In the exemplary embodiment, batter 530 is a coin cell battery.

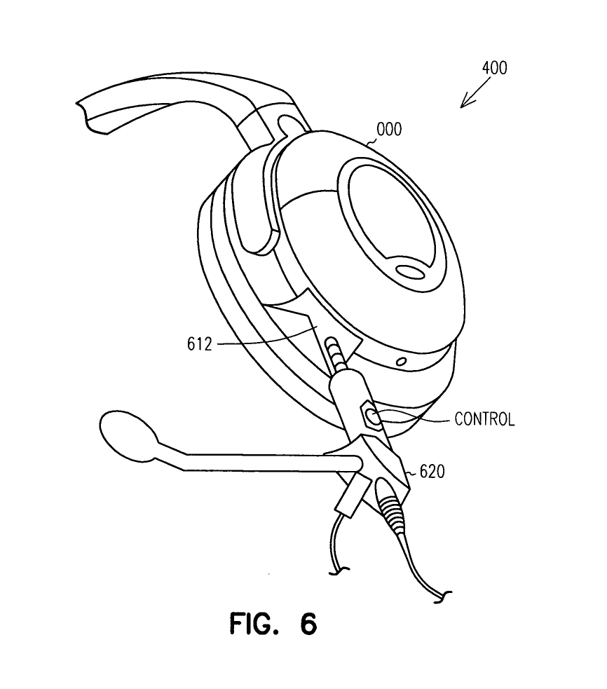

FIG. 6 shows a perspective view of an exemplary earpiece-and-headphone-microphone subassembly 600, which is applicable to systems 100 and 400. Subassembly 600 includes an earpiece 610 and a headphone-microphone connector 620. Earpiece 610 includes an sector or pie-shaped opening 612 which provides access to a socket of an audio input jack. Connector 620, which is shown with two cables, has a housing 622 with a sector or pie-shaped portion that is sized to mate with pie-shaped opening 612 in earpiece 610.

### CONCLUSION

The embodiments described above are intended only to illustrate and teach one or more ways of practicing or implementing the present invention, not to restrict its breadth or scope. The actual scope of the invention, which encompasses all ways of practicing or implementing the concepts of the invention, is defined by the following claims and their equivalents.

1. Apparatus for automatic noise reduction headphones, comprising:
a first electrical connector for connection to an audio signal input jack of automatic noise-reduction headphones;
a second male or female electrical connector coupled to the first male or female connector for connection to an audio output of a mobile communications device; and
a microphone coupled to a third male or female electrical connector for connection to an audio signal input jack of the mobile communications device.

2. The apparatus of claim 1, wherein the microphone is mounted in fixed or adjustable relation to the first male or female electrical connector.

3. The apparatus of claim 1 or 2, wherein each of the first, second, and third electrical connectors comprises a male connector.

4. A kit comprising automatic noise canceling or reducing headphones having an input for receiving electrical signals representative of acoustic energy, and a microphone for connection to an audio input jack of a mobile telephone.

5. The kit of claim 4, further comprising one or more of: an audio attenuator electrically coupled to a headphone plug; a dual-plug adaptor; an audio-connection extension cord; quarter-inch stereo phone adapter; and a battery.

6. The kit of claim 4 or 5, wherein the headphones include first and second earcups that are rotatable to facilitate storage of the headphones.

7. A hands-free accessory for a mobile communications device, such a mobile telephone, comprising:
a first connector adapted for user connection to an audio output of the mobile communications device;
a second connector electrically coupled to the first connector and adapted for user connection to an audio input port of headphones; and
a microphone coupled to a third connector adapted for user connection to a microphone input of the mobile communications device.

8. A method comprising providing a user-connectable microphone accessory for use with automatic noise reducing or canceling headphones that are not intended for use by aircraft pilots.

9. The method of claim 8, wherein the providing the microphone accessory comprises providing the accessory as an after-market add-on to the headphones.

10. The method of claim 8, wherein the providing the microphone accessory comprises providing the accessory at the point-of-sale of the headphones.
